(19) United States
(12) Patent Application Publication
(10) Pub. No.: US 2026/0041696 A1
(43) Pub. Date: Feb. 12, 2026

(54) IN-SITU PRODUCTION OF ANTI-INFLAMMATORY LIPIDS USING MILK FAT GLOBULES

(71) Applicant: THE REGENTS OF THE UNIVERSITY OF CALIFORNIA, Oakland, CA (US)

(72) Inventors: Nitin Nitin, Davis, CA (US); Ameer Taha, Davis, CA (US); Tana Hernandez Barrueta, Davis, CA (US); Mayuko Takahashi, Osaka (JP); Sadia Sattar Sultani, Pittsburg, CA (US)

(73) Assignee: THE REGENTS OF THE UNIVERSITY OF CALIFORNIA, Oakland, CA (US)

(21) Appl. No.: 19/366,402

(22) Filed: Oct. 22, 2025

Related U.S. Application Data
(63) Continuation of application No. PCT/US2024/027235, filed on May 1, 2024.
(60) Provisional application No. 63/463,192, filed on May 1, 2023.

(51) Int. Cl. A61K 31/558; A61K 9/107; A61K 31/557; C12N 9/00; C12N 9/02; C12P 7/6432; C12P 17/02

(52) U.S. Cl. CPC A61K 31/558; A61K 9/1075; A61K 31/557; C12N 9/0069; C12N 9/93; C12P 7/6432; C12P 17/02

(57) ABSTRACT

Compositions and methods are provided for producing and stabilizing oxidized lipids with anti-inflammatory properties, which are generated from polyunsaturated fatty acids as precursors to these anti-inflammatory oxylipin compounds, using intact milk fat globules extracted from milk. One process has three major steps: 1) isolation of the milk fat globules form milk; 2) incubation of the milk fat globules with polyunsaturated fatty acids; where the polyunsaturated fatty acids are encapsulated into the milk fat globule and subsequently converted to the oxidized forms such as lipid epoxides or hydroxides with potent anti-inflammatory properties, and 3) recovery of the milk fat globules which contain the anti-inflammatory lipids derived from the polyunsaturated fatty acids.

### CROSS-REFERENCE TO RELATED APPLICATIONS

This application claims priority to, and is a 35 U.S.C. § 111(a) continuation of, PCT international application number PCT/US2024/027235 filed on May 1, 2024, incorporated herein by reference in its entirety, which claims priority to, and the benefit of, U.S. provisional patent application Ser. No. 63/463,192 filed on May 1, 2023, incorporated herein by reference in its entirety. Priority is claimed to each of the foregoing applications.

The above-referenced PCT international application was published as PCT International Publication No. WO 2024/229108 A1 on Nov. 7, 2024, which publication is incorporated herein by reference in its entirety.

### STATEMENT REGARDING FEDERALLY SPONSORED RESEARCH OR DEVELOPMENT

This invention was made with Government support under grant number 2021-67017-34423 and grant number 2018-67017-27563, awarded by The National Institute of Food and Agriculture, USDA and grant number P30AG01012, awarded by National Institute of Aging, NIH. The Government has certain rights in the invention.

### NOTICE OF MATERIAL SUBJECT TO COPYRIGHT PROTECTION

A portion of the material in this patent document may be subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND

### 1. Technical Field

The technology of this disclosure pertains generally to nutrient encapsulation and delivery methods and more particularly to milk fat globule constructs with enriched oxylipin content produced in situ from selected polyunsaturated fatty acids.

### 2. Background Discussion

The health benefits associated with the consumption of polyunsaturated fatty acids (PUFAs) depends in part on the action of its enzymatically oxidized products, oxylipins. Supplementation with docosahexaenoic acid (DHA) is proposed as a nutritional intervention to address diseases associated with chronic inflammation, partially due to its enzymatically-oxidized products such as epoxydocosapentaenoic acid (EpDPE). Nevertheless, the slow in vivo turnover of DHA to EpDPE might narrow the effectiveness of this approach suggesting that that the effective delivery EpDPE directly could overcome this limitation.

Recently, the microbial production of DHA-derived oxylipins and other lipid mediators was reported. Fish oil incubated with a mixture of two recombinant Escherichia coli (expressing 8-LOX from Mus musculus and 15-LOX from Burkholderia thailandensis), subsequently converted DHA to 10-HDoHE, and Protectin Dx45. Meanwhile, a strain of Bacillus megaterium naturally expressing CYP102A1, fed with n-3 PUFAs, in vitro produced 7-HDoHE and Protectin Dx.

Consumption of a formulation containing this microorganism and the n-3 PUFAs resulted in elevated levels of 7-HDoHE in plasma; however, the question of in vivo turnover vs bioavailability has not been addressed. Accordingly, there is a need for methods producing and delivering the enzymatic production of DHA-oxylipins using non-living systems, that are simple, reliable and non-toxic.

### BRIEF SUMMARY

Compositions and methods are provided that produce and stabilize oxidized lipids with anti-inflammatory properties that have been generated from polyunsaturated fatty acids as precursors to these anti-inflammatory compounds, using intact milk fat globules extracted from mammal milk. Prophetically, this approach could be extended to various cells (microbial, plant and animal cells). There is a particular interaction between the cytochrome P450 inside of a milk fat globule (MFG) and the DHA that leads to the conversion of DHA to EpDPE that is used to illustrate the methods. The methods enable the protection/delivery of metabolically significant quantities of bioavailable DHA to the intestine, for example.

It has been shown that encapsulating DHA into milk fat globules will yield EpDPE because the membrane surrounding the globules originates from the epithelial cells in the mammary glands of the cow, which contain the enzyme cytochrome P450 that catalyzes this reaction. To demonstrate this property, DHA was first encapsulated into milk fat globules isolated from raw bovine milk, via passive diffusion at room temperature or as low as 4° C. Thereafter, the free and total oxylipins were quantified using ultra-high performance liquid chromatography coupled to tandem mass spectrometry. All five EpDPE targeted [19(20), 16(17), 13(14), 10(11) and 7(8)-EpDPE]were found to be produced. The most abundant one, 19(20)-EpDPE, reached 2.6-2.0 pmol/mg of milk cream after 30 min at the temperatures tested. It was observed that 40% was in free form and the remaining 60% was esterified (i.e. bound). It is believed that this is the first report of an active cytochrome P450 with epoxidase activity in a milk fat globule from raw bovine milk. Altogether, the results suggest the use of milk fat globules as a naturally-occurring encapsulation system capable of, upon encapsulation, converting DHA into bioactive lipids with anti-inflammatory and other properties.

Milk fat globules are colloidal systems secreted by the mammary epithelial cells and are naturally found in milk. These spherical globules, with diameters from 0.1 μm to 20 μm, encompass a lipid core surrounded by a 3-layer membrane (i.e., the milk fat globule membrane). The two outermost layers originate from the membrane of the mammary epithelial cells. Attributed to its origin, the milk fat globules from human and bovine milk contain enzymes such as xanthine oxidase and potentially other lipoxygenases as well as the Cytochrome P family of enzymes which can retain their enzymatic activity if properly handled.

One embodiment of the invention describes a composition and a method to produce enzymatically oxidized lipids with anti-inflammatory properties, which derive from polyunsaturated fatty acids (e.g., docosahexaenoic acid), using milk fat globules. The process comprises three major steps as follows: 1) isolation of the milk fat globules form milk 2) incubation of the milk fat globules with polyunsaturated fatty acids; here, the polyunsaturated fatty acids are encapsulated into the milk fat globule and subsequently converted to the oxidized forms such as lipid epoxides or hydroxides with potent anti-inflammatory properties, and 3) recovery of the milk fat globules which contain the anti-inflammatory lipids derived from the polyunsaturated fatty acids.

In another embodiment, the milk fat globules are incubated with oxylipins that are exogenously added to the milk fat globules under the same conditions along with the PUFAs. These exogenously added oxylipins are enriched at the interface of the milk fat globules compared to the core of the globule. One advantage to this approach is that it significantly stabilizes the exogenous oxylipins during oral delivery and enables the enrichment of these milk fat globules with higher levels of oxylipin content than using the DHA conversion approach alone.

These anti-inflammatory compositions can be delivered orally, topically and potentially injected at target sites to address acute and chronic inflammatory diseases. The infusion of polyunsaturated lipids in cells expressing these classes of enzymes will produce and stabilize oxidized lipids such as epoxides.

Further aspects of the technology described herein will be brought out in the following portions of the specification, wherein the detailed description is for the purpose of fully disclosing preferred embodiments of the technology without placing limitations thereon.

### BRIEF DESCRIPTION OF THE DRAWINGS

The technology described herein will be more fully understood by reference to the following drawings which are for illustrative purposes only:

FIG. 1 is a schematic diagram of a method for producing compositions of stabilized oxidized lipids with anti-inflammatory properties, which are generated from polyunsaturated fatty acids as precursors using intact milk fat globules extracted from milk according to one embodiment of the technology.

FIG. 2 is a schematic representation of a milk fat globule (MFG) and the proposed mechanism by which milk fat globules (MFG) transform exogenous docosahexaenoic acid (DHA) to its oxylipins. DHA partitions into the MFG where it is enzymatically oxidized to free oxylipins by the action of cytochrome p450 (CYP) and lipoxygenases (LOX). Free oxylipins are then esterified to phospholipids (PL) or neutral lipids (NL) by acyl-CoA synthetases and acyltransferases or hydrolyzed from them by lipases. EpDPE=epoxydocosapentaenoic acid and HDoHE=hydroxydocosahexaenoic acid. The size and location of the enzymes are schematic.

FIG. 3 is a graph of the particle size distribution of MFG from raw bovine milk, milk cream (milk after 2 cycles of centrifugal washing), and milk cream after heating treatment at 90° C. for 10 min (n=3). Results in the table are presented as mean±standard deviation; different letters indicate significant differences in MFG diameter between sample types (Tukey's test, p≤0.05).

FIG. 4 depicts graphs of percentage of free and calculated esterified (i.e., total−free) DHA-derived oxylipins from milk cream [MFG(+)] and preheated milk cream [MFG(Δ)] over 1 h incubation in the presence [DHA(+)] or absence [DHA(−)] of spiked DHA (150 μM). Results are reported as the average of n=4. DHA: docosahexaenoic acid; HDoHE=hydroxydocosahexaenoic acid; EpDPE=epoxydocosahexaenoic acid.

FIG. 5 is a graph of oxylipin concentration distributions in milk cream [MFG(+)] and preheated milk cream [MFG(Δ)] over 1 h incubation in the presence [DHA(+)] or absence [DHA(−)] of spiked DHA (150 μM).

FIG. 6 is a graph of oxylipin percentage distributions in milk cream [MFG(+)] and preheated milk cream [MFG(Δ)] over 1 h incubation in the presence [DHA(+)] or absence [DHA(−)] of spiked DHA (150 μM). Results are reported as the average of n=4. Others refers to the cumulative of arachidonic acid, eicosapentaenoic acid, alfa linolenic acid, gamma-linolenic acid, and dihommo-gamma linolenic. DHA=docosahexaenoic acid.

### DETAILED DESCRIPTION

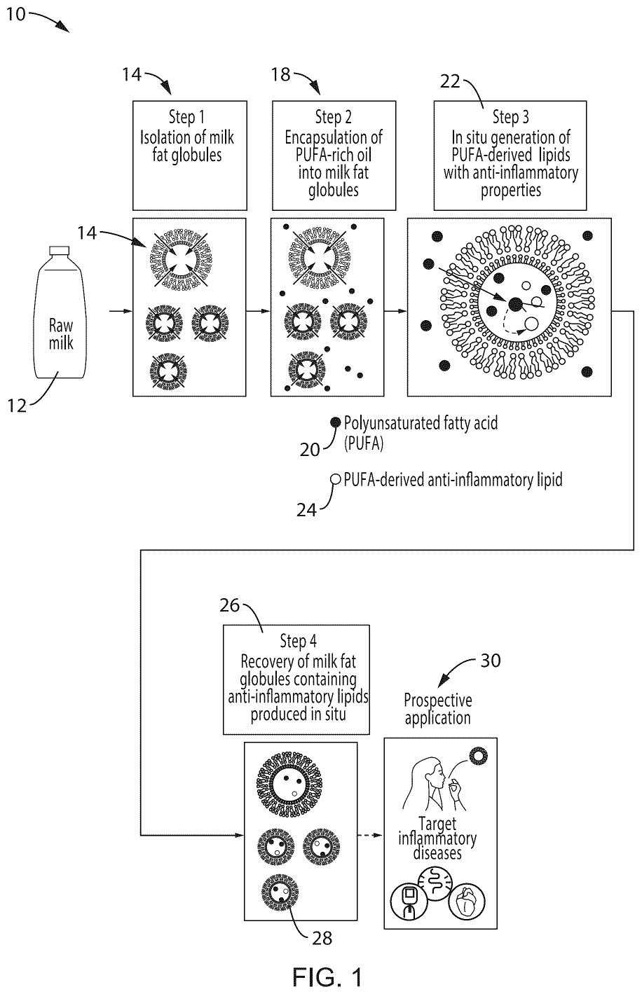

Referring more specifically to the drawings, for illustrative purposes, compositions, materials and methods for the production of milk fat globule constructs and the in situ production of desirable oxylipins from polyunsaturated fatty acids such as DHA are generally shown. Several embodiments of the technology are described generally in FIG. 1 to FIG. 6 to illustrate the characteristics and functionality of constructs and methods. It will be appreciated that the methods may vary as to the specific steps and sequence and the substrates and constructs may vary as to structural details without departing from the basic concepts as disclosed herein. The method steps are merely exemplary of the order that these steps may occur. The steps may occur in any order that is desired, such that it still performs the goals of the claimed technology.

The present technology provides novel compositions and methods to produce and stabilize oxidized lipids with anti-inflammatory properties, for example, which are generated from polyunsaturated fatty acids as precursors to these anti-inflammatory compounds, using intact milk fat globules extracted from milk.

Turning now to FIG. 1, one method 10 for producing milk fat globule constructs containing catalyzed fatty acids (e.g. oxylipins) is shown schematically. Initially, a volume of raw mammal milk 12 is acquired. Preferred milk 12 is bovine milk because it is readily available. However, goat, human or other raw milks may be used. Milk fat globules (MFG) 16 are then extracted from the raw milk 12 at block 14.

Milk fat globules 16 are complex structures that exhibit various biological activities. In one embodiment, the milk fat globules are filtered by size in the range of about 0.1 μm to about 20 μm. In another embodiment, the milk fat globules are filtered by size to be no larger than 10 μm.

It has been shown that MFGs (isolated from commercial raw and homogenized bovine milk) display the ability to catalyze the oxidation of exogenous polyunsaturated fatty acids to pro-resolving oxylipins. Docosahexaenoic acid (DHA) is used as a model molecule to illustrate the compositions and methods.

At block 18, polyunsaturated fatty acids (PUFA) are encapsulated in the extracted milk fat globules 16. Each milk fat globule 16 structure contains various metabolically active enzymes that can work cooperatively, such as CYP450, LOX, acyltransferases, and acyl-CoA synthetases. In one embodiment, the milk fat globules 16 are pasteurized prior to encapsulating the fatty acids 20.

Preferably, the polyunsaturated fatty acids (PUFA) 20 used at block 18 are one or more types of omega-3 fatty acids, omega-6 fatty acids and/or linoleic acids. In one embodiment, the fatty acid precursor is matched to a desirable oxylipin product or globular milk fat enzyme. In another embodiment, the final concentration of PUFA 20 in the milk fat globules is between about 0.05 μmol/mg to 50 μmol/mg of milk cream.

At block 22, the encapsulated polyunsaturated fatty acids are incubated for in situ generation of PUFA derived anti-inflammatory lipids 24 or other reaction products. For example, static incubation of milk cream (20% w/v in phosphate buffer and 5% ethanol) with 150 μM of DHA for 1 hour resulted in the production of approximately 13 μmol/mg cream of DHA-derived oxylipins, including those in free and esterified (i.e., bound) form. The 17-hydroxydocosahexaenoic acid [17-HDoHE] and 19(20)-epoxydocosapentaenoic acid [19(20)-EpDPE]were the most abundant reaction products that were produced. However, as many as seventy-six reaction products may be produced within the interior of the milk fat globule at block 22.

In one embodiment, the milk fat globules and PUFA are incubated at a temperature between about 4° C. to about 37° C. for a period of time to generate oxylipins.

After incubation for suitable times and conditions at block 22, the milk fat globules 28 enhanced with reaction products produced in situ are collected and purified at block 26. Because the milk cream incubated with DHA was enriched in free DHA-derived oxylipins compared to control milk cream (>80% vs <8% relative abundance, respectively), its consumption is an approach to deliver bioactive lipids, provided that the bioavailability of dietary metabolites of DHA or other reaction product is shown at block 30.

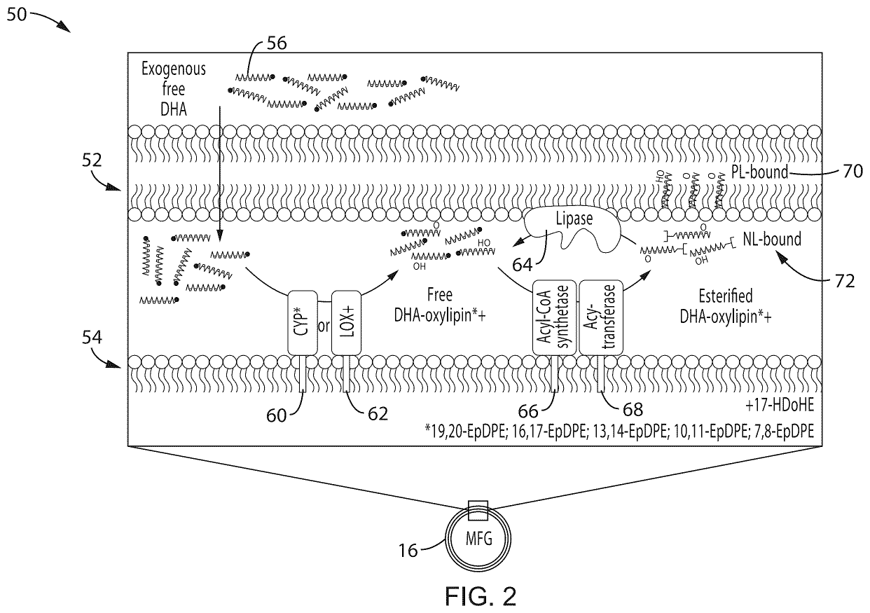

A detailed cross-sectional view of a portion 50 of a milk fat globule structure with enzymes and other active proteins to illustrate the range of possible compositions is shown in FIG. 2. The exterior of the globule has an outer double walled lipid layer 52 and an inner lipid layer 54 separated by a gap 58. Exogenous free PUFA molecules 56 that have been selected can traverse the outer layer 52 of the globule to the gap. The outer and inner layers normally have enzymes, such as cytochrome (CYP) 60, lipoxygenases (LOX) 62, Acyl-CoA synthetase or acyl-transferase 68 embedded or otherwise coupled to the inner layer 54 or may have lipases 64 bound to the outer layer 52 as shown in FIG. 2.

Catalysis or other reactions of the DHA or other polyunsaturated fatty acid substrates with surface enzymes can produce a variety of reaction products. These products may also be modified over time through interactions with different enzymes anchored in the lipid layers of the milk fat globule. For example, a DHA (docosahexaenoic acid) substrate may produce DHA-oxylipins such as HDoHE (hydroxydocosahexaenoic acid) and EpDPE (epoxydocosahexaenoic acid) in free or esterified states. The PUFA oxylipin products may also be bound to neutral lipids 72 or phospholipids 70 as illustrated in FIG. 2.

There are a number of processing condition preferences that may optimize the methods. The milk cream (rich in MFGs) is preferably between 20% to 50% w/v in buffer. The concentration of DHA and other fatty acids that may be incubated with MFGs can be varied but the milk cream is preferably incubated with DHA and other polyunsaturated fatty acids such as (e.g. EPA (eicosapentaenoic acid), ARA (arachidonic acid etc.) at a final concentration of about 1 μM to 200 μM.

The concentration of ethanol during incubation of MFGs with DHA is preferably limited. Ethanol at 5% to 10% v/v is needed during the static incubation for DHA to partition into MFGs. This addition differentiates the process from simply incubating MFGs with DHA.

The temperature that is used during incubation of MFGs with DHA can also be varied. The production of DHA-derived oxylipins that occurs when MFGs are incubated with DHA, for example, can occur in a preferred temperature range of between about 4° C. to about 37° C. The ability to generate DHA oxylipins at 4° C. is unique because many mammalian enzymes exhibit a significant reduction in enzyme activity at low temperatures. It was discovered that this is not the case for this pathway in milk (i.e. the activity of enzymes involved in oxylipin formation is retained over a wide range, enabling DHA-oxylipin synthesis under various temperature conditions). Furthermore, lowering the temperature also improves the stability of generated oxylipins.

The preferred length of time of incubation of MFGs with DHA is also a tuning feature of the methods. The production of DHA-derived oxylipins occurs when the MFGs are incubated with DHA for at least 10 minutes. The MFGs can be pasteurized (90° C. for up to 10 mins) and still retain the ability to generate DHA oxylipins. This is also an unexpected discovery because most enzyme activity is typically reduced at high temperatures.

The composition is also structurally unique. Distribution of free and esterified oxylipins in milk after incubation with DHA can be modulated. After incubating MFGs with DHA as explained above, the DHA-derived oxylipins are found as free (i.e., unbound) and esterified (i.e., bound) oxylipins. Relative to the total (i.e., free+esterified), the free oxylipin pool represents 19% to 100%, and the esterified pool is 81% to 0%, depending on the specific oxylipin. This is in contrast with native bovine MFGs, where less than 1% of oxylipins are free. This point is key because the distribution of oxylipins in the free or esterified pool directly impacts its bioavailability and in vivo activity regarding the resolution of inflammation.

The relative concentration of DHA-derived oxylipins in MFGs after incubation with DHA can also be modulated. MFGs incubated with DHA are highly enriched in free and esterified DHA-derived oxylipins, 80% to 90% and 6% to 7% of all (including non-DHA-derived) oxylipins, respectively. Conversely, in native bovine MFGs, the free and esterified DHA-oxylipins are only >8% and >0.25%, respectively. This means the MFGs prepared under the specific processing conditions described above, serve as a bio-concentrator of DHA-derived oxylipins.

In addition, the size of the MFGs could influence the generation of DHA-derived oxylipins after incubation with DHA. Comparatively smaller sizes favor the partitioning of DHA and thus enhance the production yield of DHA-derived oxylipins.

Finally, the stability of DHA-derived oxylipins in MFG during gastrointestinal delivery may be improved. Native oxylipins are highly susceptible to degradation in acidic pH. The MFG-DHA-derived oxylipins are stabilized against acidic pH. This enhanced stability is attributed to the stability of the MFG structure in acidic pH but also the localization of the DHA-derived oxylipins within the three-layer membrane of the MFG and the lipid core. The esterified form of oxylipin is predominantly associated with membrane structure and the free form may be predominantly localized in the core. The unique three-layer membrane structure of the MFG and the distribution of these generated oxylipins contribute significantly to the overall stability.

In a related construct, intact milk fat globules that have been incubated in the presence of one or more types of oxylipins as well as selected PUFAs are provided. For this incubation, oxylipins dissolved in a solvent compatible with MFG are added to the colloidal suspension of MFGs for 1 min to 2 hours. The MFGs after incubation are separated and recovered, typically using centrifugation. The infused oxylipins in these MFGs can be located both at the membrane or the lipid core region. The infusion process creates an opportunity to achieve higher concentrations of oxylipins in MFGs than that achieved with in-situ conversion of DHA alone. Similar to the above composition, the infused composition of oxylipin may also be stabilized by MFGs during oral delivery (gastric and intestinal conditions) and storage.

The resulting constructs with infused or generated oxylipins in intact milk fat globules are stabilized against degradation in both gastric and intestinal conditions with pH conditions ranging from pH 2.0 to 8.0 and temperatures ranging from about 4 to about 37° C. These constructs can be formulated into oral supplements or topical creams for therapeutic delivery and absorption of beneficial oxylipins. For example, the formulations of constructs may be used for treating inflammation in the body of a human subject by oral or topical administration of therapeutic quantities of MFG's enhanced with specific oxylipins to the subject.

The technology described herein may be better understood with reference to the accompanying examples, which are intended for purposes of illustration only and should not be construed as in any sense limiting the scope of the technology described herein as defined in the claims appended hereto.

### Example 1

To demonstrate the breadth and functionality of the constructs and methods for encapsulating PUFA's in milk fat globules and illustrative enzymatic conversions, milk fat globules were fabricated and evaluated. Milk samples were prepared from a commercial brand of raw, unhomogenized, grade A milk obtained from Jersey cows (Claravale Farm, CA) was employed in this study. For each experiment, milk was acquired from a local store (Davis Food Co-op, Davis California) and kept at 4° C. until it was used, no later than 2 days after its purchase.

Milk fat globules were isolated from the bovine milk. Briefly, the milk was diluted in PBS 1× (prepared with MilliQ water) at a 1:9 ratio, and centrifuged at 3,000×g for 5 minutes at 4° C. The cream layer was collected, resuspended at 4% (w/v) in PBS 1×, and centrifuged under the same conditions. The cream recovered from this step was used for the following experiments and will be referred to as ‘milk cream’.

To test the potential of a MFG to produce oxylipins that derive from DHA, the effect of the incubation temperature and time on their concentration and the type of oxylipins produced was evaluated. For this evaluation, one gram of milk cream was suspended in a mixture of 4.75 mL of PBS 1× and 100 μL of ethanol in a polystyrene tube, to which 150 μL of an ethanolic solution of DHA 5 mM was spiked (final concentrations: milk cream at 20% w/v, ethanol at 5% v/v, and DHA 150 μM). To account for oxylipins naturally found in milk cream, negative controls without exogenous DHA were considered.

Tubes were vortexed and allowed to incubate statically for 60 minutes, either at room temperature or on ice. Throughout the incubation, 600 μL aliquots were taken out from the same tube (i.e., repeated measurements) at times 0, 15, 30, 45, and 60 min after spiking the DHA solution (or the equivalent volume of ethanol for the negative controls), and vortexing before each sampling. Aliquots were immediately centrifuged at 3,000×g for 5 min at 4° C. and the milk cream layer was collected and stored at −80° C. for further analysis. Experiments were performed in triplicates per group. After the last sampling, extra aliquots were taken, stained with Nile red at 0.4% v/v (stock 1 mg/mL in acetone), and observed under the microscope. Since Nile red stains neutral lipids, it was used to probe the core of MFG.

### Example 2

To evaluate the effect of preheating the milk cream (before incubation with DHA) as well as the incubation time on the production of DHA-derived oxylipins, one gram of milk cream was suspended in a mixture of 4.75 mL of PBS 1×, in a glass tube. A subset of milk cream samples prepared this way were heated at 90° C. for 10 min and allowed to cool down to reach room temperature. Then 100 μL of ethanol was added to the milk cream and preheated milk cream samples, and 150 μL of an ethanolic solution of DHA 5 mM was spiked to each sample.

The final concentrations of each component were found to be the same as in Example 1. To control for oxylipins that are naturally found in MFG as well as any potential effect caused by the preheating, negative control without exogenous DHA were considered for both milk cream and preheated milk cream samples. Tubes were gently mixed by inversion and allowed to incubate statically for 60 minutes at room temperature. Then, 600 μL aliquots were taken out from the same tube (i.e., repeated measurements) at times 0 and 60 min after spiking the DHA solution (or the equivalent volume of ethanol for the negative controls), gently mixing by inversion before each sampling. Aliquots were immediately centrifuged at 3,000×g for 5 min at 4° C. and the milk cream layer was collected and stored at −80° C. for further analysis.

Particle size distribution, imaging of milk fat globules, and bacteria load determination were also evaluated. The effect of heating on the milk fat globule morphology was assessed by means of the particle size distribution and fluorescence images of the MFG membrane.

For these evaluations, one gram of milk cream was suspended in a mixture of 4.75 mL of PBS 1× (20% w/v) in glass tubes. A subset of samples was heated at 90° C. for 10 min and allowed to cool down to reach room temperature. The evaluations were performed in three independent replicates per group (‘milk cream’ and ‘preheated milk cream’).

Aliquots of 40 μL from each sample were suspended in 160 μL of PBS 1× and 1.5 μL of Oregon Green 488 DHPE (1 mg/mL in chloroform), and tubes were allowed to incubate in the dark for 10 min. This fluorescent dye probes the membrane of the MFG. Fluorescence images of the stained aliquots were collected using an inverted optical microscope (Olympus IX-7, Olympus Inc., Center Valley, PA, USA) with a 40× oil immersion objective (Olympus UPIanFL, 40×/1.30 oil, Olympus Inc., Center Valley, PA, USA). An excitation filter 480/30 nm and an emission filter 570/60 nm were used.

The microbial load in the milk before and after heating was determined by the plate counting method using plate count agar (Sigma, Cat #70152) inoculated with decimal serial dilution of the milk samples in PBS 1× and stored for 24 hours at 37° C.

The remaining samples (after removing the aliquots for imaging and bacteria counting) were diluted at a 1:1 volume ratio in 35 mM of EDTA at pH 7. The particle size distribution was quantified using a Microtrac S3500 (Microtrac instrument, US) with the following parameters: 1.46 and 1.33 refractive index of milk and water, respectively; absorbance 0.001. For the particle size, raw milk (i.e., before centrifugal separation) was also analyzed as a control.

### Example 3

For the extraction of produced oxylipins, a solution of a mixture of surrogate standards was prepared such that each of the following compounds was at a final concentration of 2 μM in methanol (LC/MS grade): d11-11(12)-EpETrE, d11-14,15-DiHETrE, d4-6-keto-PGF1a, d4-9-HODE, d4-LTB4, d4-PGE2, d4-TXB2, d6-20-HETE, and d8-5-HETE. Other solutions used were 1) antioxidant solution containing TPP, BHT, and EDTA each at 0.2 mg/mL in MilliQ/methanol 1:1 v/v, filtered through a 0.45 μm Millipore filter, 2) extraction solvent encompassing methanol with 0.1% acetic acid and 0.1% BHT and 3) solid phase extraction buffer made of 0.1% acetic acid and 5% methanol in MilliQ water. The extraction of free and total oxylipins was performed.

Extraction of free oxylipins was performed with approximately 10 mg of milk cream resuspended in 190 μL of 1×PBS, to which 600 μL of precooled methanol, 200 μL of extraction solvent, 10 μL of antioxidant solution, and 10 μL of surrogate standard solution were added. Samples were mixed by vortexing and centrifuged at 15,000×g for 10 min at 0° C. The supernatant was collected (˜0.85 mL) and MilliQ water was added to it to adjust the methanol concentration to approximately 15% v/v (˜4.5 mL). Samples then underwent solid phase extraction.

Extraction of total oxylipins was performed with approximately 10 mg of milk cream resuspended in 190 μL of 1×PBS and subjected to Folch extraction with 3 mL of chloroform/methanol (2:1 v/v) supplemented with 0.002% BHT, and 0.75 mL of 0.9% NaCl with 1 mM EDTA-2Na. Samples were vortexed vigorously and centrifuged at 598×g for 10 min at 0° C. The organic phase was collected. To the aqueous phase, 2 mL of chloroform was added, followed by vigorous vortexing and centrifugation at 598×g for 10 min at 0° C. The organic phases (containing the total lipid extract) were combined and dried under N₂ gas, reconstituted in chloroform/isopropyl alcohol (2:1 v/v), and 1 mL was dried again under N₂. To this, 200 μL of extraction buffer, 10 μL of antioxidant solution, and 10 μL of surrogate standard solution were added. Samples were hydrolyzed at 60° C. for 30 min with 200 μL of NaOH 0.4 M in MilliQ water:methanol (1:1 v/v) and allowed to cool down for 5 minutes. Following, 25 μL of acetic acid was added to acidify samples to pH 4-6 (confirmed in a few representative samples using litmus paper), followed by the addition of 1575 μL of MilliQ. Samples then underwent solid phase extraction.

Solid phase extraction of the total and free oxylipins from the samples was accomplished using 60 mg Oasis HLB columns (3 cc, Waters Corporation, CA, USA, Cat #WAT094226) prewashed with one column volume of ethyl acetate followed by two of methanol and one of solid phase extraction buffer. Samples were poured onto the column, rinsed with two volumes of solid extraction buffer, and dried under vacuum (˜15 psi, 20 minutes). Oxylipins were eluted with 0.5 mL of methanol and 1.5 mL of ethyl acetate, dried under N₂, reconstituted on 100 μL of methanol (LC/MS grade), and filtered in a Ultrafree-MC centrifugal filters (0.1 μm; Millipore Merck, Burling-ton, MA, USA, #UFC30VV00) centrifuged at 15000×g for 2 min at ° C. Samples were stored at −80° C. until analyzed by ultra-high performance liquid chromatography coupled to tandem mass spectrometry (UPLC-MC/MS).

UPLC-MS/MS analysis revealed a total of 73 oxylipins that were quantified using a 1290 Infinity ultra-high-pressure-liquid chromatography (UHPLC) system coupled to a 6460 triple-quadrupole tandem mass spectrometer (MS/MS) with electrospray ionization (Agilent Technologies, Santa Clara, CA, USA). Oxylipins were separated using a ZORBAX Eclipse Plus C18 column (2.1 mm by 150 mm; particle size 1.8 μm; Agilent Technologies, Santa Clara, CA, USA). Mobile phase A encompassed 0.1% acetic acid in MilliQ water; mobile phase B was 0.1% acetic acid in acetonitrile:methanol (85:15 v/v). The total run time was 20 min as follows. The flow gradient started with mobile phase A at 65%, and subsequently changed to 15, 0, and 65% at minutes 12, 15, and 17 respectively. During this time, the flow rate was 0.25 mL/min from 0 to 15 min, 0.4 mL/min from 15 to 19 min, and 0.30 mL/min from 17 to 20 min. Auto-sampler and column were maintained at 4 and 45° C., respectively. Meanwhile, the drying gas temperature was 300° C. with a flow of 10 L/min, sheath gas temperature was 350° C. at a flow of 11 L/min, and the nebulizer pressure was 35 psi.

The samples were injected at a volume of 10 μL, analyzed in negative electrospray ionization mode, and captured using optimized dynamic multiple reaction monitoring.

### Example 4

The results of the MFG incubation with DHA on the production of DHA-derived oxylipins were evaluated statistically. A three-way mixed ANOVA was conducted to determine the effect of incubation with or without DHA, and incubation temperature and time, on the concentration of oxylipins in MFG.

The three-way ANOVA analysis showed that the addition of DHA, but not the incubation temperature or time, significantly affected the concentration of free epoxides [19(20)-EpDPE; 16(17)-EpDPE; 13(14)-EpDPE; 10(11)-EpDPE; 7(8)-EpDPE] and mono-hydroxide (17-HDoHE) metabolites of DHA. Whereas addition of DHA only significantly affected the total oxylipins 19(20)-EpDPE and 17-HDoHE. Compared to native milk cream, incubating the cream in the presence of DHA resulted in significantly higher (p<0.001) average concentrations of these oxylipins.

The most abundant DHA-derived oxylipins both in the free and total pool were 17-HDoHE and 19(20)-EpDPE. The DHA-derived diols [19(20)-DiHDPA, 16(17)-DiHDPA]were not detected in most replicates across treatments regardless of DHA addition.

The effect of heating on the morphology of MFG from milk cream and bacterial load was also evaluated. The particle size distributions of MFG from milk, milk cream, and heated milk cream were plotted. The mean and 90th percentile diameters of MFG from milk cream were significantly higher than those found in milk (p<0.001). Subjecting the cream to heating at 90° C. for 10 min resulted in mean and 90th percentile diameters significantly smaller than the unheated cream (p<0.001) but larger compared to milk (p<0.001).

The membrane of the MFG from milk cream and heated milk cream was imaged using Oregon Green™ 488 DHPE and observed microscopically to further evaluate the structural integrity of the globules. No free fat or severe damage to the MFG membrane was observed in the samples because of the heating treatment. Milk cream had a bacterial load of 4.43±0.06 log CFU/mL. After heating, no bacterial growth was observed (detection limit 100 CFU/mL).

### Example 5

The results of preheating of milk cream (before incubation with DHA) on the production of DHA-derived oxylipins were evaluated statistically. A three-way mixed ANOVA was used to evaluate the effect of preheating the milk cream, incubation with or without DHA, and incubation time, on the concentration of oxylipins in MFG. When only the main effects were statistically significant, paired t-tests (for time effect) or t-tests were performed. The particle size distribution of the MFG in milk, milk cream, and preheated milk cream was assessed by one-way ANOVA followed by Tukey test.

The three-way ANOVA evaluation showed that incubation with DHA (but not the preheating of milk cream nor incubation time) significantly affected the concentration of DHA-derived epoxides and monohydroxide targeted, both in the free and total oxylipins pool (p<0.001). When the milk cream and preheated milk cream were incubated with DHA, these DHA-derived oxylipins were quantified at concentrations ranging from 0.7 to 3.3 and 0.7 to 5.1 μmol/mg cream, for the free and total oxylipin pool, respectively. The diols were not detected in most replicates across treatments and the most abundant DHA-derived oxylipins were 17-HDoHE and 19,20-EpDPE.

Overall, the production of DHA-derived oxylipins was observed to be maintained in preheated milk cream with morphologically preserved MFGs, upon incubation with DHA.

### Example 6

The concentration (in pmol/mg cream) of DHA-derived oxylipins from these samples were also evaluated. A total of 76 oxylipins (including non-DHA-derived) were targeted and are shown in Table 1 and Table 2 respectively.

As a trend, after 60 min of incubation with DHA, the median concentration of the DHA-derived epoxides from the total oxylipin pool was higher for milk cream compared to preheated milk cream, although their average concentration was statistically equivalent based on unpaired t-test (p >0.1).

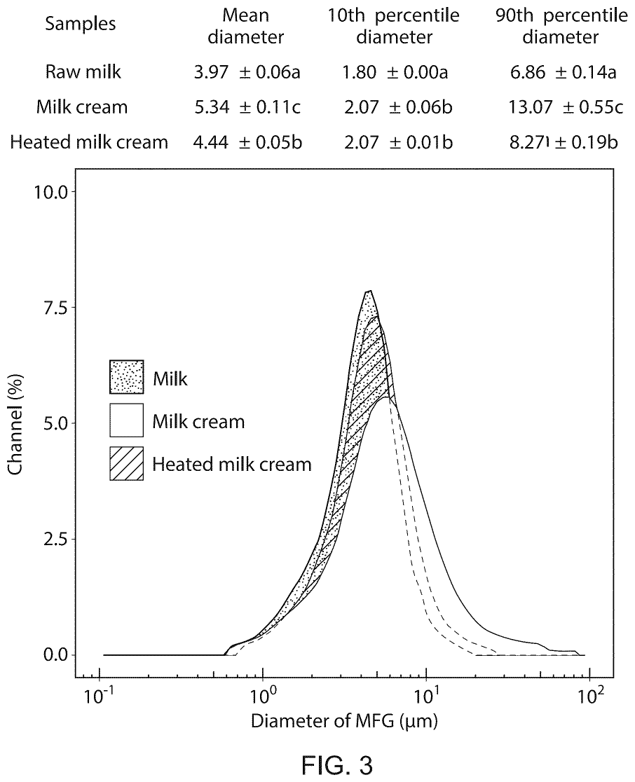

A high ratio of free to esterified DHA-derived oxylipins was shown to occur in milk cream after incubation with DHA. The ratio of free to esterified (i.e., calculated as total−free) oxylipins for the DHA-derived compounds from the preheated MFG are presented in FIG. 3.

Three-way ANOVA showed that incubation with DHA significantly affected the percentage of free oxylipins (p<0.001). The samples incubated with DHA displayed percentages of free oxylipins ranging from 19% to 100% for the epoxides, and from 60% to 80% for the monohydroxide. In these samples, unpaired t-test revealed that the percentage of free DHA-derived oxylipins at 60 min was significantly lower for the epoxides in milk cream compared to heated milk cream (p<0.06), whereas they were statistically equivalent at 0 min (p >0.2). In contrast, in control samples (without exogenous DHA) the minority of oxylipins were in the free form; the average percentages varied from 0% to 17% for the epoxides, and from 7% to 37% for the hydroxy.

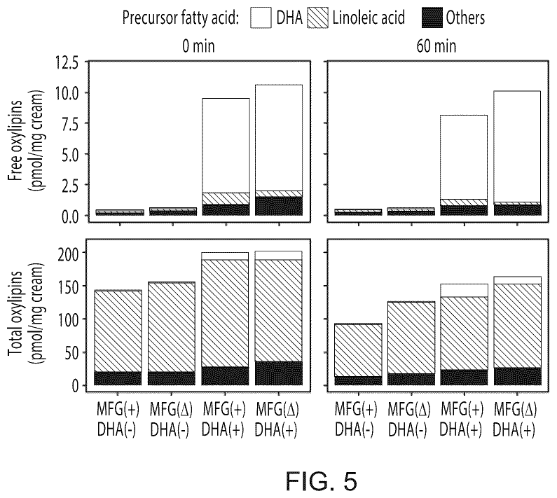

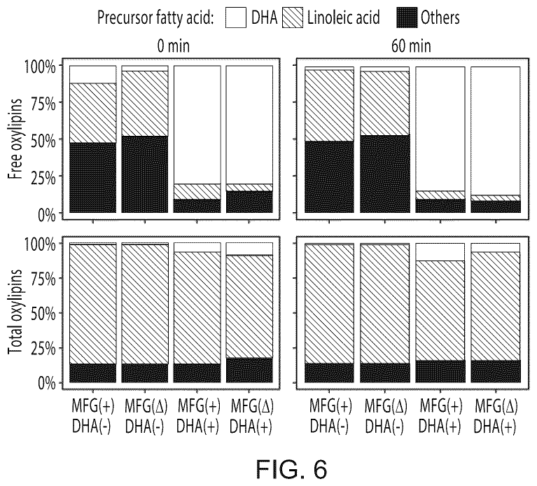

Context identification for the production of DHA-derived oxylipins from MFG relative to the oxylipins from other PUFAS was also performed. The cumulative concentration of DHA-derived oxylipins in milk cream and preheated milk cream after their incubation with DHA was compared to that of all oxylipins (including those non-derived from DHA). The cumulative concentrations were compared to that of all oxylipins are presented in FIG. 5 and FIG. 6 as concentration and relative abundance based on their precursor PUFA.

After incubation with DHA, the concentration of free oxylipins in milk cream and preheated milk cream was 8.88±1.34 and 10.40±3.70 μmol/mg cream, respectively. From these, the sum of all DHA-derived oxylipins detected was 7.27±1.21 and 8.80±2.90 μmol/mg cream, meaning that the free oxylipins whose precursor PUFA is DHA represented the majority (82.3±9.6 and 85.5±5.2%) of all free oxylipins in these samples. Conversely, the concentration of free oxylipins in control samples (without exogenous DHA) were 0.49±0.27 and 0.65±0.20 μmol/mg of cream, from which only 0.04±0.05 and 0.03±0.02 μmol/mg cream (7.9±8.9 and 4.6±3.2%) derived from DHA.

A similar trend was observed for total oxylipins. Their concentration in milk cream and preheated milk cream incubated with DHA was 199.1±36.4 and 185.5±76.8 μmol/mg cream, from which 13.4±5.3 and 12.7±7.5 μmol/mg cream derived from DHA, representing 6.6±1.9 and 6.7±1.9%. In control samples (without exogenous DHA) the concentration was 125.7±49.9 and 148.96±19.6 μmol/mg cream, but only 0.30±0.07 and 0.36±0.09 μmol/mg cream (0.27±0.08 and 0.25±0.04%) arose from DHA.

The possibility of the microbial production of DHA-derived oxylipins was also rejected since preheating the milk cream, which resulted in the inactivation of the microorganism present, had no effect on the concentration of the oxylipins compared to the milk cream. Thus, the results indicate the thermodynamic stability of the putative enzymes involved, at the experimental conditions tested (i.e., milk cream at 20% w/v). For instance, xanthine oxidoreductase (a MFGM-bound enzyme) retained more than 90% of its enzymatic activity after batch pasteurization or high-temperature short-time treatment of milk, while the enzyme was released from the MFGM at a lesser extent during centrifugation of milk diluted at 20% compared to 4% (w/v).

In summary, incubating the milk cream and preheated milk cream with DHA resulted in a 10-fold to 18-fold increase in the concentration of free DHA-derived oxylipins, and a 28-fold increase in the concentration of total oxylipins, compared to the control samples without exogenous DHA.

### Example 7

The concentrations (in pmol/mg cream) of free and esterified DHA-derived oxylipins (i.e. EpDPEs and HDoHE) produced in milk cream upon incubation with DHA were also evaluated. The concentration of DHA-derived oxylipins in milk cream and preheated milk cream incubated with DHA was 10 to 28 times higher than in control samples without exogenous DHA.

It was demonstrated that the oxylipins detected in milk cream spiked with DHA were generated by enzymes endogenous to the milk fat globules and their function was evaluated by measuring the concentrations of oxylipins from samples incubated at room temperature as compared to those performed on ice or using preheated milk cream. Contrary to expectations, these conditions had no effect on the average concentration of DHA-oxylipins. It is, however, unlikely that their synthesis resulted from autooxidation of DHA because: 1) no increase in the concentration of DHA-oxylipins was detected in the DHA stock spiked to the milk; 2) the non-enzymatic generation of these compounds occurs in a timescale of days rather than minutes, and 3) artifacts of sample preparation during the solid phase extraction were unexpected with the column that was used (Oasis-HBL).

The DHA-epoxides produced by MFG were 19,20-EpDPE, 16,17-EpDE, 13,14-EpDPE, 10,11-EpDPE, and 7,8-EpDPE. Their synthesis may involve the activity of multiple CYP450, as these regioisomers are known products of CYP450s whose gene expression have been reported in mammary glands of lactating cows: CYP2C19, CYP1A1, CYP2E1, CYP3A4, CYP2J2 16. These enzymes display metabolic rates of DHA epoxidation of 1.6, 0.5, 0.8, 0.7 and 0.2 μmol/min/pmol of CYP450, respectively. All these, except CYP2C19, preferentially oxidize the last double bond of DHA such that they predominantly generate 19,20-EpDPE, the most abundant DHA-epoxide quantified here.

Oxylipins generally exist in free form or esterified (i.e., bound) to neutral lipids and phospholipids. The esterification process depends on acyl-CoA synthetase and acyltransferase enzymes, whereas lipases catalyze their release from their bound form. Despite these three enzymes being detected in the MFGM, only the lipase activity of this fraction has been reported before.

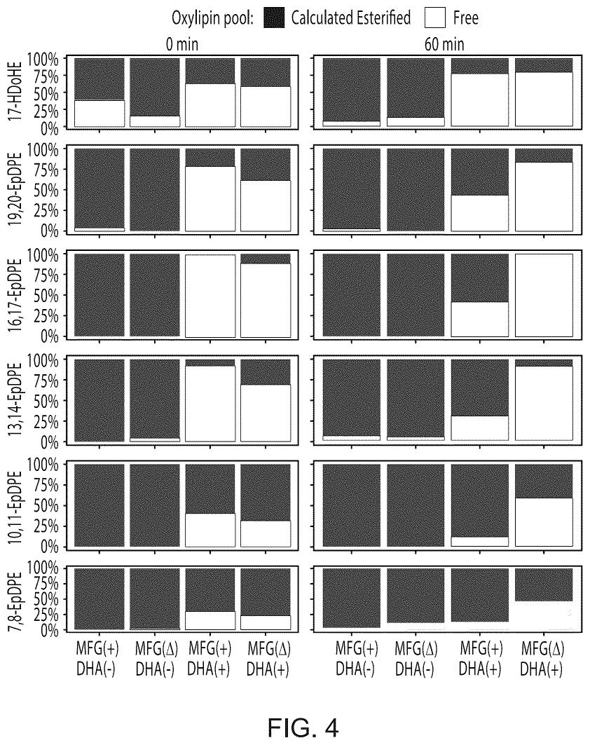

Because esterified DHA-derived oxylipins were also produced in milk cream and preheated milk after incubation with DHA, the results demonstrate the activity of these enzymes in MFG. The observation that the proportion of free oxylipins decreased at 60 minutes relative to the esterified form (FIG. 4), suggested continuing esterification of the DHA-oxylipins. The rate pool of esterification (which was impaired by heating) must be slower than the synthesis of oxylipins from their precursor fatty acid and/or their hydrolysis from their bound state since the percentage of the esterified pool was lower than the free.

Thus, the epoxidation and hydroxylation of DHA in milk cream incubated with this PUFA can be explained by the presence of metabolically active CYP450 and LOX within the MFGs. It is believed that their presence arises from the endoplasmic reticulum of the mammary epithelial cells, which represent the inner membrane of the MFGM. The CYP450 was quantified in samples from mastitic cows, while in healthy lactating ones, the gene expression of numerous isoforms was reported. The presence of these enzymes in mammary tissue has been inferred from the detection of their metabolites in milk (mostly those derived from linoleic acid), displaying a profile that differs from that of plasma.

In summary, the methods utilize the biocatalytic potential of MFGs (isolated from raw and unhomogenized mammal milk) to transform exogenous PUFAs into their oxylipins. The milk fat globules contain various metabolically active enzymes that can work cooperatively, such as CYP450, LOX, acyltransferases, and acyl-CoA synthetases. For example, the exogenous DHA in free form partitions into the MFG, where it is enzymatically oxidized to epoxides and mono-hydroxides by the action of CYP450 and LOX enzymes, respectively. Free oxylipins can be esterified to neutral lipids or phospholipids, by acyl-CoA synthetase and acyltransferases, while esterified oxylipins can be released from their bound state by lipases as illustrated in FIG. 2.

Finally, the consumption of milk cream after incubation with DHA, as presented here, could represent an approach to deliver bioactive lipids, provided that the bioavailability of dietary DHA-derived oxylipins is shown.

Embodiments of the technology of this disclosure may be described herein with reference to flowchart illustrations of methods and systems according to embodiments of the technology. Embodiments of the technology of this disclosure may also be described with reference to procedures, algorithms, steps, operations, formulae, or other computational depictions, which may be included within the flowchart illustrations or otherwise described herein. It will be appreciated that any of the foregoing may also be implemented as computer program instructions. In this regard, each block or step of a flowchart, and combinations of blocks (and/or steps) in a flowchart, as well as any procedure, algorithm, step, operation, formula, or computational depiction can be implemented by various means, such as hardware, firmware, and/or software including one or more computer program instructions embodied in computer-readable program code. As will be appreciated, any such computer program instructions may be executed by one or more computer processors, including without limitation a general-purpose computer or special purpose computer, or other programmable processing apparatus to produce a machine, such that the computer program instructions which execute on the computer processor(s) or other programmable processing apparatus create means for implementing the function(s) specified.

Accordingly, blocks of the flowcharts, and procedures, algorithms, steps, operations, formulae, or computational depictions described herein support combinations of means for performing the specified function(s), combinations of steps for performing the specified function(s), and computer program instructions, such as embodied in computer-readable program code logic means, for performing the specified function(s). It will also be understood that each block of the flowchart illustrations, as well as any procedures, algorithms, steps, operations, formulae, or computational depictions and combinations thereof described herein, can be implemented by special purpose hardware-based computer systems which perform the specified function(s) or step(s), or combinations of special purpose hardware and computer-readable program code.

Furthermore, these computer program instructions, such as embodied in computer-readable program code, may also be stored in one or more computer-readable memory or memory devices that can direct a computer processor or other programmable processing apparatus to function in a particular manner, such that the instructions stored in the computer-readable memory or memory devices produce an article of manufacture including instruction means which implement the function specified in the block(s) of the flowchart(s). The computer program instructions may also be executed by a computer processor or other programmable processing apparatus to cause a series of operational steps to be performed on the computer processor or other programmable processing apparatus to produce a computer-implemented process such that the instructions which execute on the computer processor or other programmable processing apparatus provide steps for implementing the functions specified in the block(s) of the flowchart(s), procedure (s) algorithm(s), step(s), operation(s), formula(e), or computational depiction(s).

It will further be appreciated that the terms “programming” or “program executable” as used herein refer to one or more instructions that can be executed by one or more computer processors to perform one or more functions as described herein. The instructions can be embodied in software, in firmware, or in a combination of software and firmware. The instructions can be stored locally to the device in non-transitory media or can be stored remotely such as on a server, or all or a portion of the instructions can be stored locally and remotely. Instructions stored remotely can be downloaded (pushed) to the device by user initiation, or automatically based on one or more factors.

It will further be appreciated that as used herein, the terms controller, microcontroller, processor, microprocessor, hardware processor, computer processor, central processing unit (CPU), and computer are used synonymously to denote a device capable of executing the instructions and communicating with input/output interfaces and/or peripheral devices, and that the terms controller, microcontroller, processor, microprocessor, hardware processor, computer processor, CPU, and computer are intended to encompass single or multiple devices, single core and multicore devices, and variations thereof.

From the description herein, it will be appreciated that the present disclosure encompasses multiple implementations of the technology which include, but are not limited to, the following:

A composition of matter comprising intact milk fat globules that have been incubated in the presence of one or more types of polyunsaturated fatty acids (PUFA); wherein said incubated intact milk fat globules have increased levels of one or more oxylipins relative to unincubated milk fat globules.

The composition of any preceding or following implementation, wherein said one or more types of polyunsaturated fatty acids (PUFA) is selected from the group of omega-3 fatty acids, omega-6 fatty acids and linoleic acids.

The composition of any preceding or following implementation, wherein the polyunsaturated fatty acid comprises docosahexaenoic acid (DHA) and the increased oxylipin comprises epoxydocosapentaenoic acid (EpDPE) or hydroxydocosahexaenoic acid (HDoHE).

The composition of any preceding or following implementation, wherein a source of the milk fat globules is selected from the group of from bovine, human and goat milk sources.

The composition of any preceding or following implementation, wherein the milk fat globules have a diameter in the range of about 0.1 μm to about 20 μm.

A method for generating oxylipins, the method comprising: (a) providing a plurality of milk fat globules and one or more types of polyunsaturated fatty acids (PUFA); (b) encapsulating the one-or-more types of fatty acids within an interior of the milk fat globules, the interior having a membrane interface; (c) generating PUFA derived oxylipins within the interior of each of the plurality of milk fat globules; and (d) recovering milk fat globules containing generated oxylipins produced in situ.

The method of any preceding or following implementation, wherein the one or more types of polyunsaturated fatty acids (PUFA) is selected from the group of omega-3 fatty acids, omega-6 fatty acids and linoleic acids.

The method of any preceding or following implementation, further comprising matching an oxylipin product with a fatty acid precursor and a globular milk fat enzyme.

The method of any preceding or following implementation, further comprising filtering the milk fat globules by size.

The method of any preceding or following implementation, wherein the milk fat globules filtered by size are in the range of about 0.1 μm to about 20 μm.

The method of any preceding or following implementation, wherein the milk fat globules filtered by size are no larger than 10 μm.

The method of any preceding or following implementation, wherein the milk fat globules are pasteurized prior to encapsulating the fatty acids.

The method of any preceding or following implementation, wherein the final concentration of PUFA in the milk fat globules is 0.05 to 50 μmol/mg of milk cream.

The method of any preceding or following implementation, wherein the milk fat globules and PUFA are incubated at between 4° C. to 25° C. to generate oxylipins for a period.

The method of any preceding or following implementation, further comprising producing a supplement of therapeutic concentrations of recovered milk fat globules containing oxylipins.

The method of any preceding or following implementation, further comprising purifying the generated oxylipins from the recovered milk fat globules.

The method of claim 6, wherein the PUFA derived oxylipins comprise one or more of 19,20-EpDPE, 16,17-EpDPE, 13,14-EpDPE, 10,11-EpDPE, 7,8-EpDPE and 17-HDoHE.

A method for concentrating specific anti-inflammatory lipids, the method comprising: providing at least one PUFA fatty acid that is a precursor to an anti-inflammatory lipid; providing a plurality of mammal milk fat globules; incubating the PUFA fatty acids with the milk fat globules for a time period; and wherein the PUFA fatty acid precursors are converted to the anti-inflammatory lipids by the milk fat globules.

The method of any preceding or following implementation, wherein the PUFA comprises docosahexaenoic acid (DHA), Eicosapentaenoic acid (EPA), or Arachidonic acid (ARA) and the anti-inflammatory lipid is one or more of 19,20-EpDPE, 16,17-EpDPE, 13,14-EpDPE, 10,11-EpDPE, 7,8-EpDPE, 17-HDoHE, 17(18)-EpETE, 14(15)-EpETE, 11(12)-EpETE, 8(9)-EpETE, 11(12)-EpETrE, 8(9)-EpETrE, [5(6)-EpETrE.

The method of any preceding or following implementation, wherein the milk fat globules with PUFA are incubated at 4° C. to 25° C. to generate anti-inflammatory lipids for a period of time.

The method of any preceding or following implementation, further comprising matching an anti-inflammatory lipid with a fatty acid precursor and a globular milk fat enzyme.

The method of any preceding or following implementation, further comprising incubating the milk fat globules in the presence of one or more types of oxylipins.

The method of any preceding or following implementation, wherein the oxylipins and the anti-inflammatory lipids of the MFGs are stable in pH conditions ranging from pH 2.0 to pH 8.0.

A method for treating gut inflammation in a human subject where such treatment comprises oral administration to the subject of the composition of matter or the anti-inflammatory lipid generated by any method herein.

A composition of matter comprising intact milk fat globules that have been incubated in the presence of one or more types of oxylipins.

A composition of matter in which infused or generated oxylipins in intact MFGs are stabilized against degradation in both gastric and intestinal conditions.

A composition of matter in which infused or generated oxylipins are stable in pH conditions ranging from pH 2.0 to 8.0.

As used herein, the term “implementation” is intended to include, without limitation, embodiments, examples, or other forms of practicing the technology described herein.

As used herein, the singular terms “a,” “an,” and “the” may include plural referents unless the context clearly dictates otherwise. Reference to an object in the singular is not intended to mean “one and only one” unless explicitly so stated, but rather “one or more.”

Phrasing constructs, such as “A, B and/or C”, within the present disclosure describe where either A, B, or C can be present, or any combination of items A, B and C. Phrasing constructs indicating, such as “at least one of” followed by listing a group of elements, indicates that at least one of these groups of elements is present, which includes any possible combination of the listed elements as applicable.

References in this disclosure referring to “an embodiment”, “at least one embodiment” or similar embodiment wording indicates that a particular feature, structure, or characteristic described in connection with a described embodiment is included in at least one embodiment of the present disclosure. Thus, these various embodiment phrases are not necessarily all referring to the same embodiment, or to a specific embodiment which differs from all the other embodiments being described. The embodiment phrasing should be construed to mean that the particular features, structures, or characteristics of a given embodiment may be combined in any suitable manner in one or more embodiments of the disclosed apparatus, system, or method.

As used herein, the term “set” refers to a collection of one or more objects. Thus, for example, a set of objects can include a single object or multiple objects.

Relational terms such as first and second, top and bottom, upper and lower, left and right, and the like, may be used solely to distinguish one entity or action from another entity or action without necessarily requiring or implying any actual such relationship or order between such entities or actions.

The terms “comprises,” “comprising,” “has”, “having,” “includes”, “including,” “contains”, “containing” or any other variation thereof, are intended to cover a non-exclusive inclusion, such that a process, method, article, apparatus, or system, that comprises, has, includes, or contains a list of elements does not include only those elements but may include other elements not expressly listed or inherent to such process, method, article, apparatus, or system. An element proceeded by “comprises . . . a”, “has . . . a”, “includes . . . a”, “contains . . . a” does not, without more constraints, preclude the existence of additional identical elements in the process, method, article, apparatus, or system, that comprises, has, includes, contains the element.

As used herein, the terms “approximately”, “approximate”, “substantially”, “substantial”, “essentially”, and “about”, or any other version thereof, are used to describe and account for small variations. When used in conjunction with an event or circumstance, the terms can refer to instances in which the event or circumstance occurs precisely as well as instances in which the event or circumstance occurs to a close approximation. When used in conjunction with a numerical value, the terms can refer to a range of variation of less than or equal to ±10% of that numerical value, such as less than or equal to ±5%, less than or equal to ±4%, less than or equal to ±3%, less than or equal to ±2%, less than or equal to ±1%, less than or equal to ±0.5%, less than or equal to ±0.1%, or less than or equal to ±0.05%. For example, “substantially” aligned can refer to a range of angular variation of less than or equal to ±10°, such as less than or equal to ±5°, less than or equal to ±4°, less than or equal to ±3°, less than or equal to ±2°, less than or equal to ±1°, less than or equal to ±0.5°, less than or equal to ±0.1°, or less than or equal to ±0.05°.

Additionally, amounts, ratios, and other numerical values may sometimes be presented herein in a range format. It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth.

The term “coupled” as used herein is defined as connected, although not necessarily directly and not necessarily mechanically. A device or structure that is “configured” in a certain way is configured in at least that way but may also be configured in ways that are not listed.

Benefits, advantages, solutions to problems, and any element(s) that may cause any benefit, advantage, or solution to occur or become more pronounced are not to be construed as a critical, required, or essential feature or element of the technology described herein or any or all the claims.

In addition, in the foregoing disclosure various features may be grouped together in various embodiments for the purpose of streamlining the disclosure. This method of disclosure is not to be interpreted as reflecting an intention that the claimed embodiments require more features than are expressly recited in each claim. Inventive subject matter can lie in less than all features of a single disclosed embodiment.

The abstract of the disclosure is provided to allow the reader to quickly ascertain the nature of the technical disclosure. It is submitted with the understanding that it will not be used to interpret or limit the scope or meaning of the claims.

It will be appreciated that the practice of some jurisdictions may require deletion of one or more portions of the disclosure after the application is filed. Accordingly, the reader should consult the application as filed for the original content of the disclosure. Any deletion of content of the disclosure should not be construed as a disclaimer, forfeiture, or dedication to the public of any subject matter of the application as originally filed.

All text in a drawing figure is hereby incorporated into the disclosure and is to be treated as part of the written description of the drawing figure.

The following claims are hereby incorporated into the disclosure, with each claim standing on its own as a separately claimed subject matter.

Although the description herein contains many details, these should not be construed as limiting the scope of the disclosure, but as merely providing illustrations of some of the presently preferred embodiments. Therefore, it will be appreciated that the scope of the disclosure fully encompasses other embodiments which may become obvious to those skilled in the art.

All structural and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

TABLE 1
76 Oxylipins Measured by UPLC-MS/MS

| Precursor | | Associated |
|---|---|---|
| fatty acid | Oxylipin | Enzyme |
| DHA | Resolvin D1 | LOX |
| | (±)19,20-dihydroxy-4Z,7Z,10Z,13Z,16Z- | CYP/sEH |
| | docosapentaenoic acid | |
| | (±)16,17-dihydroxy-4Z,7Z,10Z,13Z,19Z- | CYP/sEH |
| | docosapentaenoic acid | |
| | 17-hydroxydocosahexaenoic acid | LOX |
| | 19(20)-epoxydocosapentaenoic acid | CYP |
| | 16(17)-epoxydocosapentaenoic acid | CYP |
| | 13(14)-epoxydocosapentaenoic acid | CYP |
| | 10(11)-epoxydocosapentaenoic acid | CYP |
| | 7(8)-epoxydocosapentaenoic acid | CYP |
| EPA | Resolvin E1 | COX |
| | Prostaglandin E3 | COX |
| | Prostaglandin D3 | COX |
| | 8,15-dihydroxyeicosatetraenoic acid | LOX |
| | 5,15-dihydroxyeicosatetraenoic acid | LOX |
| | 17,18-dihydroxyeicosatetraenoic acid | CYP |
| | 14,15-dihydroxyeicosatetraenoic acid | CYP |
| | 11,12-dihydroxy-5Z,8Z,14Z,17Z- | CYP |
| | eicosatetraenoic acid | |
| | 8,9-dihydroxy-5Z,11Z,14Z,17Z- | CYP |
| | eicosatetraenoic acid | |
| | 5,6-dihydroxyeicosatetraenoic acid | CYP |
| | Leukotriene B3 | LOX |
| | 15-hydroxyeicosapentaenoic acid | LOX |
| | 8-hydroxyeicosapentaenoic acid | LOX |
| | 12-hydroxyeicosapentaenoic acid | LOX |
| | 5-hydroxyeicosapentaenoic acid | LOX |
| | 17(18)-epoxyeicosatetreaenoic acid | CYP |
| | 14(15)-epoxyeicosatetreaenoic acid | CYP |
| | 11(12)-epoxyeicosatetreaenoic acid | CYP |
| | 8(9)-epoxyeicosatetreaenoic acid | CYP |
| DGLA/ | Prostaglandin E1 | COX |
| GLA | Prostaglandin D1 | COX |
| | 15(S)-hydroxyeicosatrienoic acid | LOX |
| ALA | 9-hydroxyoctadecatrienoic acid | LOX |
| | 13-hydroxyoctadecatrienoic acid | LOX |
| ARA | 20-COOH-Leukotriene B4 | LOX |
| | 6-keto-prostaglandin F1 alpha | COX |
| | 20-OH-Leukotriene B4 | LOX |
| | Tromboxane B2 | COX |
| | Prostaglandin F2 alpha | COX |
| | Prostaglandin E2 | COX |
| | Prostaglandin D2 | COX |
| | Leukotriene D4 | LOX |
| | Lipoxin A4 | LOX |
| | Leukotriene E4 | LOX |
| | Prostaglandin J2 | COX |
| | Prostaglandin B2 | COX |
| | Leukotriene C4 | LOX |
| | 6-trans-leukotriene B4 | LOX |
| | Leukotriene B4 | LOX |
| | 14,15-dihydroxyeicosatrienoic acid | CYP |
| | 11,12-dihydroxyeicosatrienoic acid | CYP |
| | 8,9-dihydroxyeicosatrienoic acid | CYP |
| | 15-deoxy-Prostaglandin J2 | COX |
| | 20-hydroxyeicosatetraenoic acid | CYP-H |
| | 5,6-dihydroxyeicosatrienoic acid | CYP |
| | 15-hydroxyeicosatetraenoic acid | LOX |
| | 15-oxo-eicosatetraenoic acid | LOX |
| | 11-hydroxyeicosatetraenoic acid | LOX |
| | 9-hydroxyeicosatetraenoic acid | LOX |
| | 12-hydroxyeicosatetraenoic acid | LOX |
| | 8-hydroxyeicosatetraenoic acid | LOX |
| | 12-oxo-eicosatetraenoic acid | LOX |
| | 5-hydroxyeicosatetraenoic acid | LOX |
| | 14(15)-epoxyeicosatrienoic acid | CYP |
| | 5-oxo-eicosatetraenoic acid | LOX |
| | 11(12)-epoxyeicosatrienoic acid | CYP |
| | 8(9)-epoxyeicosatrienoic acid | CYP |
| | 5(6)-epoxyeicosatrienoic acid | CYP |
| LA | 9,12,13-trihydroxyoctadecamonoenoic acid | LOX |
| | 9,10,13-trihydroxyoctadecamonoenoic acid | LOX |
| | 12,13-dihydroxyoctadecamonoenoic acid | CYP/sEH |
| | 9,10-dihydroxyoctadecamonoenoic acid | CYP/sEH |
| | 13-hydroxyoctadecadienoic acid | LOX |
| | 13-oxo-octadecadienoic acid | LOX |
| | 13-oxo-octadecadienoic acid | LOX/PGH |
| | 9-oxo-octadecadienoic acid | LOX/PGH |
| | 12(13)-epoxyoctadecamonoenoic acid | CYP |
| | 9(10)-epoxyoctadecamonoenoic acid | CYP |

TABLE 2
Non-DHA-derived Oxylipins Measured by UPLC-MS/MS

| | | MRM |
|---|---|---|
| Oxylipin Name | Abbreviation | Transition |
| d4-6-keto-prostaglandin F1 alpha | d4-6-keto-PGF1a | 373.3 −> 167.1 |
| d4-Tromboxane B2 | d4-TXB2 | 373.3 −> 173.2 |
| d4-Prostaglandin E2 | d4-PGE2 | 355.2 −> 275.3 |
| d4-Leukotriene B4 | d4-LTB4 | 339.2 −> 197.2 |
| d11-14,15-dihydroxyeicosatrienoic | d11-14,15-DiHETrE | 348.2 −> 207.1 |
| acid | | |
| d6-20-hydroxyeicosatetraenoic acid | d6-20-HETE | 325.2 −> 281.2 |
| d4-9-hydroxyoctadecadienoic acid | d4-9HODE | 299.2 −> 172.3 |
| d8-5-hydroxyeicosatetraenoic acid | d8-5-HETE | 327.2 −> 116.1 |
| d-11-11(12)-epoxyeicosatrienoic | d-11-11(12)EpEtrE | 330.2 −> 167.2 |
| acid | | |

What is claimed is:

1. A composition of matter comprising:
intact milk fat globules that have been incubated in the presence of one or more types of polyunsaturated fatty acids (PUFA);
wherein said incubated intact milk fat globules have increased levels of one or more oxylipins relative to unincubated milk fat globules.

2. The composition of claim 1, wherein said one or more types of polyunsaturated fatty acids (PUFA) is selected from the group of omega-3 fatty acids, omega-6 fatty acids and linoleic acids.

3. The composition of claim 2, wherein said polyunsaturated fatty acid comprises docosahexaenoic acid (DHA) and said increased oxylipin comprises epoxydocosapentaenoic acid (EpDPE) or hydroxydocosahexaenoic acid (HDoHE).

4. The composition of claim 1, wherein a source of said milk fat globules is selected from the group of from bovine, human and goat milk sources.

5. The composition of claim 1, wherein the milk fat globules have a diameter in the range of about 0.1 μm to about 20 μm.

6. A method for generating oxylipins, the method comprising:
(a) providing a plurality of milk fat globules and one or more types of polyunsaturated fatty acids (PUFA);
(b) encapsulating the one-or-more types of fatty acids within an interior of said milk fat globules, said interior having a membrane interface;
(c) generating PUFA derived oxylipins within the interior of each of the plurality of milk fat globules; and
(d) recovering milk fat globules containing generated oxylipins produced in situ.

7. The method of claim 6, wherein said one or more types of polyunsaturated fatty acids (PUFA) is selected from the group of omega-3 fatty acids, omega-6 fatty acids and linoleic acids.

8. The method of claim 6, further comprising:
matching an oxylipin product with a fatty acid precursor and a globular milk fat enzyme.

9. The method of claim 6, further comprising: filtering the milk fat globules by size.

10. The method of claim 9, wherein the milk fat globules filtered by size are in the range of about 0.1 μm to about 20 μm.

11. The method of claim 9, wherein the milk fat globules filtered by size are no larger than 10 μm.

12. The method of claim 6, wherein the milk fat globules are pasteurized prior to encapsulating the fatty acids.

13. The method of claim 6, wherein the final concentration of PUFA in the milk fat globules is 0.05 to 50 μmol/mg of milk cream.

14. The method of claim 6, wherein the milk fat globules and PUFA are incubated at between 4° C. to 25° C. to generate oxylipins for a period.

15. The method of claim 6, further comprising:
producing a supplement of therapeutic concentrations of recovered milk fat globules containing oxylipins.

16. The method of claim 6, further comprising:
purifying the generated oxylipins from the recovered milk fat globules.

17. The method of claim 6, wherein the PUFA derived oxylipins comprise one or more of 19,20-EpDPE, 16,17-EpDPE, 13,14-EpDPE, 10,11-EpDPE, 7,8-EpDPE and 17-HDoHE.

18. A method for concentrating specific anti-inflammatory lipids, the method comprising:
providing at least one PUFA fatty acid that is a precursor to an anti-inflammatory lipid;
providing a plurality of mammal milk fat globules;
incubating said PUFA fatty acids with said milk fat globules for a time period; and
wherein said PUFA fatty acid precursors are converted to said anti-inflammatory lipids by said milk fat globules.

19. The method of claim 18, wherein the PUFA comprises docosahexaenoic acid (DHA), Eicosapentaenoic acid (EPA), or Arachidonic acid (ARA) and said anti-inflammatory lipid is one or more of 19,20-EpDPE, 16,17-EpDPE, 13,14-EpDPE, 10,11-EpDPE, 7,8-EpDPE, 17-HDoHE, 17(18)-EpETE, 14(15)-EpETE, 11(12)-EpETE, 8(9)-EpETE, 11(12)-EpETrE, 8(9)-EpETrE, [5(6)-EpETrE.

20. The method of claim 18, wherein the milk fat globules with PUFA are incubated at 4° C. to 25° C. to generate anti-inflammatory lipids for a period of time.

21. The method of claim 18, further comprising:
matching an anti-inflammatory lipid with a fatty acid precursor and a globular milk fat enzyme.

22. The method of claim 18, further comprising:
incubating said milk fat globules in the presence of one or more types of oxylipins.

23. The method of claim 22, wherein said oxylipins and said anti-inflammatory lipids of the MFGs are stable in pH conditions ranging from pH 2.0 to pH 8.0.

24. A method for treating gut inflammation in a human subject where such treatment comprises oral administration to the subject of the composition of matter of claim 1.

25. A method for treating gut inflammation in a human subject where such treatment comprises oral administration to the subject of the anti-inflammatory lipid generated by the method of claim 18.
